(19) United States
(12) Patent Application Publication
(10) Pub. No.: US 2006/0147556 A1
(43) Pub. Date: Jul. 6, 2006

(54) Process and apparatus for modifying plant extracts

(76) Inventor: Graeme Brewer, Sunshine Coast (AU)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/559,303

(22) PCT Filed: Jun. 17, 2004
(86) PCT No.: PCT/AU04/00790

§ 371 Date: Dec. 2, 2005

(30) Foreign Application Priority Data
Jun. 18, 2003 (AU) 2003903058

(51) Int. Cl. A61K 36/18

(52) U.S. Cl. 424/725

(57) ABSTRACT

Phytonutrients or other active materials can be extracted from plant material using a particular process which requires extracting juice from the plant material, followed by breaking open the plant cells to release the phytonutrients by subjecting the juice to high shear conditions, followed by concentrating the juice using a membrane which is preferably a nanofiltration membrane, and collecting the concentrated extract which can then be spray dried info a powder and used as a nutritional supplement, or which can be further extracted using supercritical carbon dioxide to provide an even more concentrated product. The process is designed to maintain a high levels of phytonutrients in the active condition and to reduce plant and equipment costs especially in the use of supercritical carbon dioxide.

### FIELD OF THE INVENTION

The present invention is directed to a process that enables concentrated phytonutrients to be obtained from plant material, and a product made by the process. The present invention is also directed to a method that enables concentrated phytonutrients to be obtained in a cheaper manner. The present invention is also directed to a general process that enables concentrated plant extracts to be obtained.

### BACKGROUND ART

Eating a balanced diet is essential for disease prevention, and to maximise the benefits of a balanced diet, quantity and variety in the fruits and vegetables eaten is important. Fruits and vegetables contain different combinations of vitamins and minerals and other compounds called phytonutrients. Research is now providing evidence that health benefits of fruits and vegetables are also due these phytonutrients.

Plant extracts are gaining increasing demand for their therapeutic value. These compounds are also referred to as phytochemicals or phytonutrients. Phytonutrients can include nutritional substances in foods which act as important biological response modifiers. Examples are carotenoids, the red-orange pigments that give fruits and a vegetables their distinctive colour. Many phytochemicals are anti-oxidants that protect the body against the damaging effect of oxygen free radicals. Many phytochemicals are converted by the body to vitamin A, which is used in the immune system as well as to prevent blindness.

Scientists are recognizing that a great many factors in foods contribute to their ability to act as modifiers of biological function. The use of specific whole foods and their concentrates, along with the traditional vitamin and mineral factors, provides a much more powerful influence on normalizing biological function and functional health than the traditional diet of processed foods with the addition of a simple vitamin/mineral supplement with antioxidants.

There is a large range of plants that contain phytonutrients. These include a wide range of fruits, vegetables, spices, herbs and also other plants that are not usually consumed in their natural state such as barley grass, alfalfa, algae and medicinal herbs. Examples include grapes, pineapples, oranges, blueberries, apples, cranberry, papaya, aloe vera, acerola, tomato, carrot, beetroot, broccoli, spinach, chilli, ginger, alfalfa, beetroot, olives, green tea, panax ginseng, lecithin (soy derived), inulin, and ginkgo biloba. Commercial fruit concentrates also contain phytonutrients and these concentrates can contain red grape, pineapple, orange, blueberry, apple, cranberry, papaya, aloe vera, and acerola.

Phytonutrients are also found in many grass products which include wheat grass, barley grass, alfalfa grass and spirulina.

However, the level of phytochemicals in plants is not large and a great quantity of plant material must be consumed to benefit from the therapeutic properties of the phytochemicals or other beneficial compounds present in the plants.

For this reason it is known to extract the “actives” such as the phytochemicals and other beneficial components from plant material to provide a concentrate.

It is very important that high quality plant extracts for medicinal and nutritional use are produced and which have a high level of the therapeutically active compounds.

A well known process for producing herbal extracts and phytopharmaceuticals involves harvesting of the herbal plants, drying the plants, and then extracting active components from the plants with a solvent. The dried plants are ground to a fine chaff and placed into columns. A solvent percolates through the bed of dried plants in the column, is collected and the solvent is removed to provide the concentrate.

The use of dried herbs or dried plants as a starting point in preparation of a concentrate has some disadvantages. The main disadvantage is that the plant deteriorates during the drying process, and this results in a reduction of “actives” that can be removed, and also allows the concentrate to contain deteriorated actives that can be a source of contamination in the final concentrate and that can be difficult to remove from the concentrate. It is found that as soon as the fresh plant is cut, the high moisture level allows the immediate multiplication of microbes, bacteria and fungi. The enzyme systems in these microbes start breaking down the phytochemicals in the plants which can also include the pharmacologically active compounds. The natural enzyme systems in the plants themselves also begin the process of breaking down the plant phytochemicals as soon as the plants are harvested.

Another disadvantage is that the cost of drying can be substantial in terms of labour and capital equipment. Most plants in their fresh state consist of approximately 90% moisture, so there is a large amount of water to be removed. Drying costs can be reduced by windrowing in the fields for a few days, but this can only increase the level of breakdown in the plant material and leaves the plants exposed to contamination.

Another disadvantage is that dried herbs or other plants may be stored for a relatively long period of time (up to six months) prior to being sold to processes for solvent extraction. This can contribute further to the decomposition of pharmacologically active compounds in the plant material.

Surveys of retail dried herbal products purchased from health shops show a wide range of therapeutic activities, and this is probably caused by the conventional drying process. It is not desirable to have a product (e.g. dried herbs) having varying levels of activities as this can lead to dosage errors.

The conventional method of concentrating the dried plant material by solvent extraction also suffers from disadvantages. Solvent extraction is carried out on ground dried plant material (e.g. dried and ground herbs) by allowing the particular solvent to percolate through a bed of ground plant material in a column. Due to the low bulk density of the plant material in the column, large volumes of solvents are required to extract the actives (e.g. phytochemicals). Also there is a high proportion of inert plant fibre that has no therapeutic value that takes up room in the column. At the end of the extraction this bulky herb residue must be compressed to recover solvent, so there can be considerable losses of expensive solvents.

Another problem with conventional solvent extraction in the use of ground dry plants in a column and using solvent, is the limited yield. The plant material cannot be ground too fine, as the solvent will not percolate through the bed (usually under gravity). Because the plant material must be relatively coarse (fine chaff), the penetration of the solvent is limited so yields of active are low and larger volumes of solvent are required to improve extraction. However, if the plant material is not finely ground, the plant cells will not break open to release the phytonutrients from the plant cells. Therefore, this current extraction method requires a balance between grinding sufficiently to at least partially open the plant cells but not grinding so finely that it becomes almost impossible to pass solvents through a column containing the ground plant material. This process is therefore inherently inefficient.

Another problem with large packed columns is channelling, where some plant material is under-extracted and other parts over-extracted resulting in low yields and use of high amounts of solvents.

Obviously, it is desirable to extract the plant material using non toxic solvents. However many active components in the plant material have low solubility in non toxic solvents (e.g. water or a water/ethanol mixture). For this reason rather toxic solvents such as N-hexane, acetone, methanol and methylene chloride are used to extract therapeutic actives. There is always concern about the amount of toxic solvent residue that remains after distilling the solvent from the extract. These organic solvents are toxic, many are highly flammable and pose a very high fire risk and can also be quite expensive.

It is also known to extract actives from plant material using supercritical carbon dioxide as the solvent. This process involves filling a column with ground dried plant material and pumping supercritical liquid carbon dioxide though the column at very high pressure (200-400 Bar).

The low bulk density of the ground herbs used to pack the column requires that the dimensions for the column be large to accommodate commercial production i.e. 200-300 litres. However the vessel must withstand 200-400 Bar so the vessel has to be engineered very strong and made of stainless steel, due to the corrosive nature of low pH of liquid carbon dioxide, which makes for a very high capital cost. The large dimension of the vessel also requires large amounts of carbon dioxide, so these two factors make for a very expensive extraction process. Also larger diameter columns reduce the efficiency of plug flow through. The large volume of carbon dioxide used requires that the must be recycled around the system in a closed loop which means further high capital cost of refrigeration equipment and a bulk liquid carbon dioxide tank. A further drawback in current process is that volatile components extracted from the plants can be retained in the carbon dioxide when it evaporates in the separation tank. This means that these extracted volatile materials build up in the carbon dioxide in the recirculation loop. This also contaminates the carbon dioxide for use in extraction from the next plant species used in the extractor.

In the current form used supercritical carbon dioxide extraction is a very expensive process so it can only be applied to very high value plant extracts. A reference article (Fine Chemicals 1998) compares the pay back rate in months for various extraction systems.

| | |
|---|---|
| Hexane | 9 months |
| Steam | 37 months |
| Super critical carbon dioxide | 540 months |

This high cost makes the extraction of only very high value active components a commercial proposition.

However, supercritical carbon dioxide extraction is otherwise a very good method to concentrate the actives in plant material, so there would be a great advantage if the cost of supercritical carbon dioxide extraction could be reduced both in operating and capital cost.

There would also be a great advantage if it were possible to provide a plant concentrate that did not require a long drying process prior to extraction with a solvent.

There would also be a great advantage if it were possible to product a dried flowable solid product (e.g a powder) from the concentrate and that has a good level of actives.

There would also be a great advantage if it were possible to provide a process to prepare a concentrate and which reduced the need for toxic organic solvents and which could lower the volume of solvent required.

It will be clearly understood that, if a prior art publication is referred to herein, this reference does not constitute an admission that the publication forms part of the common general knowledge in the art in Australia or in any other country.

### OBJECT OF THE INVENTION

It is an object of the invention to provide a process which provides a concentrate extracted from plant material and which may overcome at least some of the above-mentioned disadvantages or provide a commercial or useful choice.

In one form, the invention resides in a method to provide a concentrate obtained from plant material, the method comprising at least partially extracting juice from the plant material, subjecting at least some of the extracted juice to shear conditions to at least partially open plant cellular material, and removing at least some of the water to provide a concentrate.

It can be seen that this method does not require the use of plant material that has been dried for long periods of time and therefore the starting material is not appreciably deteriorated compared to plant material that has been dried for a long time. Of course, the invention can use dried plant material if desired but need not be limited to the use of dried plant material. For instance, it may be possible to reconstitute dried plant material by mixing with water.

The plant material will typically comprise plant material that contains phytonutrients or other desirable active material. Suitable plant material will be as described above. The plant material may comprise leaf material, stem material, tubers, root material the entire plant, part of the plant and the like. However, if it is known that the phytonutrients are overwhelmingly present in a certain part of the plant (e.g. in the tuber with respect to carrots) then it is preferred that only this part of the plant is used in order to avoid inefficiencies.

The plant material can be initially collected by any suitable means or by any harvesting means. This may include manual harvesting, automated harvesting etc. It is preferred that the plant material is processed as quickly as possible after being harvested in order to prevent deterioration of the plant material that has been described above. Typically, the delay between harvesting and processing should be only a few hours and ideally should be less than two hours. However, it may be possible to subject the harvested plant material to low temperatures in order to prevent or to reduce deterioration and thereby enabling the delay to be somewhat larger. For instance, it may be necessary to call the harvested plant material to between 3°-10°. This can be done at any suitable means including cold water, cold air etc.

Suitably, the harvested plant material is subject to a sterilising step prior to extraction of the juice. The sterilising step may be conducted using a bactericide. A suitable bactericide may comprise ozone. However, the invention should not be limited only to the use of this particular bactericide.

The liquid, or juice can be extracted from the plant material by any conventional means. This may comprise the use of pressure to extract plant juice from the plant. However, no particular limitation needs to be placed on the invention by the method by which the liquid is removed from the plant material. The plant material will typically be chopped, crushed etc to assist in removal of the plant juice. It is preferred that as much juice as possible is removed from the plant material. Thus, it is preferred that between 20%-99% of the juice is removed from the plant material.

This crushing and extraction process may release some of the phytonutrients, but many phytonutrients will still be locked away in intact cell structures in the juice.

It is preferred that the juice is screened to remove plant fibres, and other nonsoluble material. The screen may comprise a relatively coarse screen to remove only the relatively cause plant fibres and nonsoluble material.

The juice, or at least part of the juice is then treated to conditions of high shear. There are commercial machines that will subject the juice to conditions of high shear. One commercial machine is known as the SILVERSON machine. In this particular machine, the juice is subject to intense hydraulic shear. The shear conditions will open up more of the plant cell structure is to release phytonutrients which would be otherwise locked away. The above commercial machine has been identified as an example of a machine that can provide conditions of high shear, but no particular limitation is meant thereby, and invention is not to be limited only to this type of machine to provide the high shear conditions to the extracted liquid/juice.

The treated juice (that is the juice that has been treated to the shear conditions) is then concentrated to form a concentrate. It is preferred that the concentration is carried out using a semipermeable membrane process as this does not require the use of large amounts of heat. A particularly preferred membrane separation process is nanofiltration which can remove up to 80% of the water. An advantage of using a nanofiltration membrane is that the membrane as well as removing the water will also remove salts such as potassium chloride.

It is preferred that the concentrated material is dried into a solid flowable material (such as a powder). This can be done using a conventional drying process which requires the use of heat, but the amount of heat required may be considerably less due to the initial concentration of the juice.

Prior to, or after drying the concentrated material, it is preferred that the concentrated material is subjected to an extraction step. The extraction step may comprise solvent extraction. The solvent may comprise supercritical carbon dioxide which has been described above.

The extracted material from the solvent extraction step will typically be high in phytonutrients, and this material can then be dried into a flowable material.

### BRIEF DESCRIPTION OF THE DRAWINGS

FIGURE 1. Illustrates a general flowchart

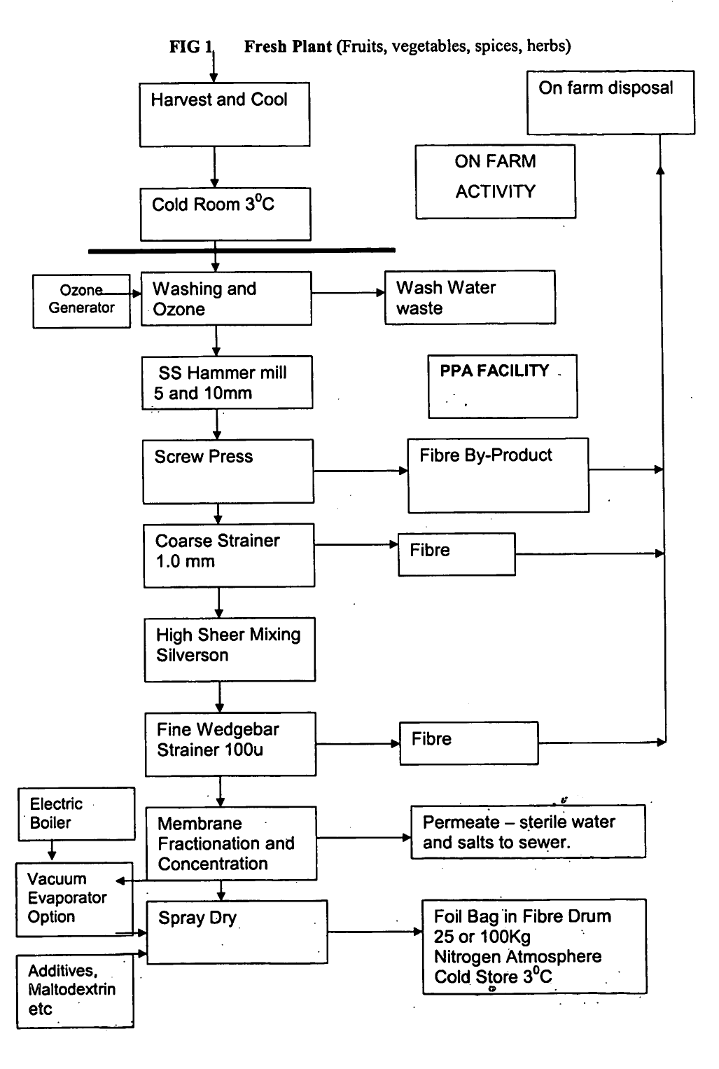

### DETAILED DESCRIPTION

General Process Description

Stage 1

The invention relates to a process for extracting active nutraceutical and phytopharmaceutical phytochemicals from plants using the fresh plants, or dried plants reconstituted with water where no fresh plant is available.

The plants are harvested and brought to the processing facility to provide a short “harvest to extraction time” i.e. less than two hours. If the weather is warm or there is a transport time of greater than two hours, the harvested plants are cooled to 3-10° C. in the field. At the factory the harvested plant material is held in a cold room.

The plant material is first washed and sterilised using a bactericide in the washing water.

The plant material is spun dry and ground in a milling unit that feeds to an extraction press where the plant juice is extracted under pressure. The fibre from the press can be blended with water and more juice extracted.

After screening through a strainer to remove the coarse fibre, the juice, which is not a true solution but rather a fine suspension of extracted plant, material is submitted to high shear mixing to release the contents of the chloroplast and plant cells.

This solution is then pumped through a membrane cartridge, preferably nanofiltration, which has the capacity to remove 80% of the water. The membrane rejects molecules above approximately 300 daltons and has low rejection of monovalent salts such as potassium and chloride, which are the main salts in plant extracts.

Another advantage of using nanofiltration membranes for concentration is that that the low rejection of the nanofiltration membrane for salts reduces the increase in osmotic pressure of the solution as it is concentrated. This allows the membranes to remove significant quantities of water an reach concentration factors of better than 5 on the therapeutic active components that are of most interest.

If further concentration is required this can be achieved at low temperature using vacuum evaporation. Concentration by membrane is much lower cost than vacuum evaporation.

A dry extract can then be produced by low temperature spray drying. Because only a small amount of the original water is left to be removed in the spray drying stage (in the order of 5%) only a small spray drier is required. Fine atomisation of the feed into the spray drying process gives evaporation in a fraction of a second and the dried particles are cooled due to the evaporative cooling effect. Operating with dehumidified drying air can reduce the operating temperature.

This powdered product can be sold in a powdered form or formulated into dose forms such as capsules, tablets, gels and the like or incorporated into topical formulations.

Where a more refined extract is required the retentate from nanofiltration can be processed using microfiltration. The permeate from a microfilter with a pore size of 0.2 microns will produce a sterile product.

Further refinement and concentration of therapeutic active components can be achieved by using the spray dried powder from Stage 1 as the feed stock for a variety of STAGE 2 solvent extraction processes.

Stage 2—Solvent Extraction

In a further embodiment the spray dried powder from Stage 1 can be extracted using super critical fluid extraction with carbon dioxide or alternatively extraction with a range of organic solvents.

The SCF-CO2 process is a liquid/solid extraction process with carbon dioxide under high pressure acting as the solvent. Due to the large reduction in the volume of plant material achieved by using the spray dried powder from STAGE 1 as the feed stock instead of the original dried plant material the size of the high pressure vessel for super critical extraction can be reduced by 60 to 80%.

In a further embodiment the spray dried powder can be extracted by the standard array of organic hydrocarbon solvents. However, the quantity of solvents used is substantially reduced and the capital expenditure on column is much lower. The organic hydrocarbon solvents can be distilled off to produce an oleo resin. The organic hydrocarbon solvent can also be removed by using solvent resistant nanofiltration membrane process. In this process the solvent extract is pumped through a nanofiltration membrane cartridge at high pressure in the range of 10 to 30 bar.

Stage 3

In a further embodiment the oleo resin from STAGE 2 can be further extracted using a liquid/liquid super critical fluid extraction column using super critical carbon dioxide as the solvent.

In a further embodiment, the SCF, CO2 extract can-be used in nutraceutical products, phytopharmaceutical and cosmeceutical compositions suitable for use by humans.

In a further embodiment of the invention the SCF, CO2 extract from STAGE 2 can be further refined to isolate particular phytochemicals by the use of solvent extraction using a range of organic solvents from the group—hexane, methanol, acetone, ethanol and methylene chloride.

In a further embodiment the organic solvent extract from stage 2 can be further refined to phytochemicals using columns for adsorption, ion exchange or chromatographic separation.

In a further embodiment the product from the stage 3 Liquid/liquid SCF CO2 process can be refined by the use of adsorbents, ion exchange, chromatographic separation. In a further embodiment the CO2 separation vessel from the liquid/liquid SCF CO2 column can be preceded by a packed column containing an adsorbent, ion exchange resin or chromatographic resin.

In a further embodiment the SCF CO2 from STAGE 2 can be further refined using SCF Chromatography. In this process SCF CO2 is used as the carrier solvent in the chromatographic column. The chromatographic column operates at high pressure and the column is packed with the usual chromatographic packing such as C18.

### GENERAL EXAMPLES

The extraction process is in two stages:

Stage 1—Aqueous extraction from the fresh plant through to a spray dried powder.

Stage 2 —Solvent extraction of the spray dried powder from stage 1 to produce an extract with very high concentrations of Phytochemicals

This stage 2 solvent extraction can either use SCF CO2 which is the preferred embodiment or and solvent.

According to the invention, the plants for extraction are harvested either by machine or hand and are transported immediately to the processing facility. If the time from harvest to processing is going to be greater than two hours, then the harvested plants are field cooled rapidly to between 3° C. and 10° C. by circulating chilled water over them. Another known process for rapid cooling of plant material is a vacuum cooling chamber. Commercial units are available that can cool produce to 2° C. within 30 minutes.

Plant material can be held in cold stores at the processing facility if there is a delay in processing.

The extraction process starts with the washing of the plant material using high pressure water sprays followed by a holding bath with dissolved ozone. The ozone bath is maintained at 2-6 mg per litre of dissolved ozone, with the optimal level being 4 mg of ozone per litre.

The plant material is spun dry to remove excess water and passes to a milling unit. This unit can be a commercial hammer mill or cutter grinder. It is considered that a skilled person will understand the type of milling unit that can be used to crush the plant material.

The milled plant material passes to a press to extract the juice. There is quite a range of commercial presses that can perform the task, such as a roller mill, screw press, belt press and air bag press. It is considered that a skilled person will understand the type of presses or other devices that can be used to extract juice from the plant material.

The extracted juice passes over a strainer to remove larger plant particles that have been extracted with the juice. The screen opening is between one and four millimetres, with the preferred opening being two millimetres. It is considered that a skilled person will understand the type of screens or other types of filters that can be used to remove large plant particles that have been extracted with the juice.

To improve extraction the fibre left from the first press can be mixed with water and then passed through the press again to yield a diluted juice, which can be combined with the first pressings.

The filtered juice is then subject to high shear. A number of commercial units are available for this process. The high shear can be imparted by an homogeniser or Silverson mixer (Trade Mark). An homogeniser forces the liquid stream through very fine openings to create high shear. A Silverson mixer stirs the liquid stream and draws it past impeller rotating at high speed with narrow clearance between the outside cage. It is considered that a skilled person will be able to understand the type of machine or device that can be used to imparted a high shear on the filtered juice.

The extracted juice is not a true solution but includes a suspension of fine plant particles such aggregates of plant cells and chloroplasts. It appears that the high shear at least partially breaks down the chloroplast and plant cells and releases their contents into solution thereby increasing the availability of active compounds.

This treated juice is filtered using a strainer with an opening of 50 to 500 microns with a preferred opening of 100 microns. A commercial unit most suited for this is a rotating stainless steel wedge bar strainer with self cleaning back flush. The back flush can be high pressure water or steam. It is considered that a skilled person will understand the type of strainer that can be used to filter the treated juice.

The filtered juice then passes to the membrane separation plant. The membrane plant is fitted with a commercial spiral wound nanofiltration membrane as suplied by Osmonics Corp U.S.A. The pore size of the membrane is selected depending on the size of the molecules to be rejected by the membrane. In the preferred embodiment a nanofiltration membrane has a rejection or from 150 to 500 daltons . Suitable commercial membrane cartridges are spiral wound cartridges. The pore size of these membranes is 5 to 20 angstroms with a preferred pore size of 10 angstroms. The membranes operate at high crossflow and a transmembrane pressure of 10 to 40 Bar with a preferred operating pressure of 25 to 35 Bar.

The membrane has a low rejection for water and monovalent salts such as potassium and chloride. These salts pass through the membrane with the water as the permeate. Ninety-eight percent of the organic molecules above 300 daltons are retained by the membrane and there is still a high rejection down to 200 daltons.

The majority of organic molecules above 200 daltons are the ones showing therapeutic properties. These can include the following non limiting examples:

Carotenoids Beta-carotene, Alpha-carotene, Lycopene, Lutein, Zeaxanthin

Flavonoids: Quercitin, Rutin, Anthocyanins, Catechins, Procyanidins, Coumarin

Phytoestrogens:

Isoflavones genistein, daidzein, glycitein, Coumestans, Lignans, Resveartrol

Glucosinulates:

sulforphane

Allylic compounds:

Diallyl sulphide

Terpenoids

Phenolic acids

Cholorophyll

Many of the organic molecules of pharmacological interest are 5 to 10 times this size so there is almost 100% retention of the most desired components. Examples of these large molecules are flavanoids, isoflavones, polysaccharides, polyphenols, carotenes, and glycosides of smaller ring compounds.

This membrane separation stage can concentrate the liquid extract by a factor of four to six times. A solids level from 15% to 20% can be achieved in the membrane retentate.

This membrane concentrated extract can be spray dried at this concentration or concentrated further by vacuum evaporation to a concentration of 30% to 40% solids depending on the viscosity of the concentrate which can vary depending on the species of plant being extracted. Commercial vacuum evaporators used for this type of concentration can be flash evaporators or an Alfalaval spinning cone vacuum evaporator, which operate at low temperature and evaporate at approximately 45° C. to 50° C. vapour temperature.

The concentrated extract is then dried to a powder using a low temperature spray drying technique. The commercial spray drier used for this stage is a small tower spray drier using air atomisation. Air atomisation gives very fine particle size and rapid equilibrium. It is considered that a skilled person will understand the technique required to convert the concentrated extract into a flowable solid products such as a powder.

The incoming air has been dehumidified so drying can take place at low temperature. Incoming air temperature is in the range of 100 to 200° C. The rapid evaporation of water from the very fine atomised particles causes adiabatic cooling so that the product particles are not heated and the enzyme systems and therapeutically active components in these particles retain all of their activity. With this technique even heat sensitive products can be dried.

This spray dried powder is the raw material for the stage 2 extraction process; however these powders can be marketed as a separate product in their own right.

The dried powder can be marketed as a nutraceutical, herbal medicine or nutritional supplement. The product is suitable to be formulated into the following dose forms-powder, capsules and tablets. An advantage with this product is that the membrane separation process removes most of the potassium chloride. Potassium chloride is deliquescent (absorbs moisture from the atmosphere) and it has been observed with powder nutritional supplements from other manufacturers that have not been through our membrane process, the powder in retail packs soon goes lumpy after the bottle has been opened due to absorption of moisture from the atmosphere. Also moisture absorption can be a problem as microbes can grow when sufficient moist has been absorbed.

These manufacturers add quantities of starch a filler to try and mop up this absorbed moisture to keep their product free flowing. This addition of a filler is undesirable because larger doses are required because of dilution of the actives.

Where higher levels of actives are required in a botanical extract or phytopharmaceutical or phytochemical the dried powder product can be used as the feedstock for more sophisticated downstream extraction techniques.

Second Stage Extraction

The next stage of extraction that can be used is a batch type liquid/solid extraction using supercritical carbon dioxide as the solvent.

Supercritical fluid extraction operates by pumping supercritical CO2 through the solid ground plant to be extracted at very high pressure in a column. The compounds extracted by the supercritical CO2 depend on the pressure applied to the column. More volatile compounds such as essential oils are extracted at low pressures. Other compounds such as carotenoids are extracted using higher pressures. That is, the lipophylic solvent properties of the supercritical carbon dioxide can be increased by increasing the pressure in the column. The higher the pressure the more lipophilic the solvent properties of the carbon dioxide become. The critical point for carbon dioxide is 31° C. and 73 ATM. SCF extraction operates at moderate temperatures 35 to 400° C. Operating pressures for essential oils can be down to 100 ATM and higher molecular weight compounds such as carotenoids around 250 to 300 ATM. The CO2 extract passes through a reducing value and into a separation tank where CO2 evaporates to a gas leaving the extracted therapeutic actives behind in the separation tank. Pressure reduction is usually down to 40 to 60 ATM.

By preparing a spray dried powder from the fresh plant the volume of solids to be extracted is substantially reduced and can be in the order of five times smaller than what would be required if dried whole plants were used in the column.

These extraction columns are fabricated from stainless steel and operate at very high pressures; 200 bar to 500 bar, so large columns are very expensive to build. A five fold reduction in the size of a column can give very substantial reduction in capital costs for an SCF (supercritical fluid) plant. Large columns and bed depths result in poorer distribution of solvent and hence give lower yield.

One of the main operating costs for SCF is the cost of liquid carbon dioxide solvent. The much smaller column in this invention uses substantially less carbon dioxide.

Another major capital cost is a CO2 recovery system. This may not be required if low volumes of CO2 are used in the smaller SCF column.

In a further embodiment the powder from stage one used as feed to the SCF, CO2 column can be pelletised into fine extruded rod a about 1 mm diameter.

In a further embodiment the spray dried powder from stage one can have a small amount of excipient added such as methyl cellulose. The excipient added can be any of a large number of inert polymers that will not be dissolved by the supercritical carbon dioxide. This inert frame work supports the column packing while the carbon dioxide dissolves the active therapeutic actives out of the matrix. This mixture can be compressed into small tablets or formed into beadlets using a coating device. The SCF CO2 extractor can then have the bed constructed as a radial flow bed with reduced path length and high capacity for the small column.

In another embodiment of the invention the powder from the spray drier can be extracted by non polar organic solvents. The choice of solvent varies depending on the species of plant being extracted or the type of pharmacological active being extracted. The following is the range of organic solvents used for this type of extraction: -n-hexane, acetone, methanol and methylene chloride.

In existing processes the organic solvent extraction takes place on the original ground dry herb. The ground plant is filled into columns and the organic solvent percolates through the bed.

In this embodiment the spray dried powder already contains high concentrations of the actives. The organic solvent extraction can take place in a much smaller vessel and substantially less organic solvent is required.

Because the powder is very fine the extraction can take place in a stirred tank reactor and a very high recovery of therapeutic active can be achieved. The solvent extract is filtered from the residue and the solvent removed by vacuum distillation.

Because in this process much less organic solvent is used therefore the distillation equipment can be much smaller and operating costs are lower due to the substantial reduction in expensive organic solvents used. The organic solvents are toxic and highly flammable so the smaller quantities used make for a much safer commercial process.

The product from many of these organic solvent extractions result in an oleo resin after the solvent has been evaporated under vacuum. Oleo resins can be used in a range of products and have a high level of actives.

Further extraction of the oleo resin can be carried out by using liquid/liquid supercritical fluid extraction in a continuous column using supercritical CO2. The oleo resin is pumped into the top of the column through a pressure reducing valve to a separation tank where the CO2 evaporates away leaving the refined product with higher level of actives in the product.

Analyte (waste) passes out at the base of the column. The products extracted from the oleo resin depend on the operating pressure of the SCF column.

Higher pressures are required to dissolve the higher molecule weight products required. Volatile products can be SCF extracted by CO2 at around 200 bar high molecular weight products can be extracted at 250 to 300 Bar.

A preferred embodiment of the present invention can include the following steps:

Short harvest to extraction time for fresh plant,

Sterilising the plants by washing with ozone saturated water,

Pressing out the fresh juice,

Breaking open the plant cells and choroplasts in the juice with high shear mixing,

Fine straining of the juice,

Concentrating using nanofiltration membranes to concentration factor of five,

Removal of monovalent ions and water in the nanofiltration membrane permeate,

Optional further concentration by vacuum evaporation,

Low temperature spray drying of the concentrate to form a powder,

Agglomerating the powder with a high molecular excipient such as methyl cellulose,

Further extracting the spray dried solids by liquid/solid supercritical fluid extraction with CO2 to produce a refined product with a high level of actives,

Operating the liquid/solid SCF CO2 a sequential range of pressures to produce various fractions of extract containing different therapeutic actives,

Alternatively adding a modifier to the CO2 such as ethanol,

Alternatively, in another embodiment, extracting the spray dried solids with a range of organic solvents to produce oleo resins with a high level of actives,

Downstream extraction of the oleo resins using liquid/liquid SCF with CO2 in a continuous column to produce highly refined products with high level of actives.

The aqueous extraction of the fresh plant along with the concentration and fractionation by nanofiltration membrane can be carried out with high volumes plant material with safety and low cost. The spray drier required is only small since most of the water has been removed by membrane at low cost.

The large reduction on solids from fresh plant to the spray dried powder allows for a dramatic reduction in the size of the down stream extraction units required, namely:

liquid/solid SCF extraction

organic solvent extraction

In the process where aqueous extraction of the fresh plant is followed by:

high shear mixing

membrane processing

spray drying

agglomeration with a polymer

liquid/solid SCF extraction with CO2

We have a process where in the preferred embodiment no undesirable organic solvents have been used in the extraction process. The organic solvents are toxic and highly flammable and require special facilities. There is always concern about the level of residue left in the product from organic solvents. International regulations are being introduced to limit the amount of organic solvent residue.

If the plant source is from organic certified crops then this process would be the only extraction process of this type that could be certified as organic. Ie. The cleanest, greenest process for extraction of nutraceuticals and phytopharmaceuticals.

The resulting products have the higher levels of therapeutic actives than any other process because there is no losses when the plants are dried. Very high yields are obtained by using high sheer to open up the chloroplasts and plant cells.

### Specific Example 1

### Chilli

A crop of cayenne chilli was harvested and delivered to the processing facility within one hour.

100 kg of chilli was washed with high pressure water and then submerged in a wash bath containing water at 20 degrees celsius with dissolved ozone at a concentration of 5 mg per litre. The chilli was drained and then milled in a stainless steel hammer mill.

The milled material passed into a screw press and the juice was extracted. Large solids 1-2 mm were removed in a wedge bar strainer with a clearance of I The juice was pumped to a 100 litre tank and blended with high sheer using a Silverson mixer. The blended juice was then passed through a fine wedge bar strainer with a clearance of 100 microns.

The juice was then pumped at high pressure (2500 Kpa) through a nanofiltration spiral wound membrane cartridge. The membrane was a spiral wound nanofiltration cartridge from Osmonics U.S.A. with a nominal pore size of 10 angstroms. (10 000 angstroms=1 microns)

The operating temperature was 25 degrees Celsius. A concentration factor of 5 was achieved. The permeate from the membrane process contained mainly water and monovalent ions -chloride, sodium and potassium. The concentrate was spray dried in a small tower spray drier using air atomisation. The drying air was dehumidified by refrigeration. The yield of spray dried powder from the original 100 kg of chilli fruits was 6 kg.

The spray dried powder was agglomerated by blending with methyl cellulose and drying in a fluid bed drier. The agglomerated matrix was loaded into a 10 litre batch operated liquid/solid supercritical fluid CO2 extractor. Supercritical liquid Carbon Dioxide was passed through the column at 35 degrees Celsius and 220 bar pressure.

The liquid carbon dioxide extract passed through a reduction valve and into a separation tank at 50 bar. The CO2 extract was warmed to 35 C and the CO2 evaporated leaving a residue with a high level of capsaisin the main active phytopharmaceutical

### Specific Example 2

### Solvent Extraction of Chilli

The spray dried powder from Example 1 was extracted by solvent extraction in a small stirred tank reactor. The solvent used was acetone.

The acetone was removed from the extract by vacuum evaporation to produce an oleo resin. This oleo resin was extracted in a small stirred tank reactor. The solvent used in this stage was ethanol. The ethanol was removed from the extract by vacuum distillation to yield a second, more refined, oleo resin with high levels of capsaicin.

This oleo resin can be used at a 10% concentration in pressure sprays for personal protection, riot control and disarming criminals. The oleoresin can also be used as concentrated chilli flavour in foods.

The oleo resin from this stage can be further refined by using a liquid/liquid supercritical fluid extraction using CO2.

The oleo resin was pumped into near the top of the small stainless steel SCF column. Supercritical carbon dioxide was pumped in near the bottom of the column and passed counter current to the oleo resin flow.

The extracted product passed out of the top of the column and after the pressure reducing valve the CO2 evaporated to leave a high purity product residue of capsaicin. To meet United States Pharmacopoeia standards, the product must contain 70% of capsaisinoids. The column operated at 35 degrees Celsius and 200 bar.

### Specific Example 3

### Red Clover

A crop of red clover was harvested and rapidly cooled in field using a vacuum cooling chamber. The cooled red clover was taken to the factory and held in cold storage at −2 degrees C.

100 kg of red clover was washed with high pressure water and then passed through a bath of water with dissolved ozone at a concentration of 5 mg per litre.

The red clover was drained and passed through a stainless steel hammer mill with a 10 mm screen. The milled material passed into a screw press and the juice was extracted. The extracted juice passed through a strainer to remove particles above 2 mm. The juice then passed through a high sheer Silverson mixer to release the contents of cells and chloroplasts.

This product them passed through a fine 100 micron wedgebar screen. This filtered juice was then concentrated and modified using nanofiltration membrane which removed water and monovalent ions. Chloride, sodium, potassium. The juice was further concentrated to 30% solids in a vacuum evaporator. This concentrate was then dried in a tower spray drier using air atomisation. The resulting spray dried powder had a moisture content of below 5%.

This powder product could be marketed in a powder form as a rich source of isoflavones.

The powder was agglomerated with methylcellulose in a fluid bed drier and fed into the vessel of a supercritical extractor. Supercritical carbon dioxide was pumped through the vessel and then passes through a reducing valve and into the separation tank.

A product separated out in the separation tank that was very rich in the isoflavones—genistein, daidzein, formonontein and biochanin.

### Specific Example 3

### Red Grapes

A fresh supply of red grapes was purchased and washed in a commercial Tipax washer manufactured by Tripax Engineering, Victoria Australia. The product is drawn down deep into the tank by means of a vortex created by angled underwater jets. The washed grapes spill over to a dewatering vibrator and up a conveyor into the hammermill and then into a screw press.

Red juice is extracted solids are retained by a 0.7 mm screen.

The juice is subjected to high sheer in a Silverson mixer and then passed through a 100 micron strainer to a nanofiltration membrane plant fitter with a nanofiltration spiral wound cartridge. The membrane is operated with a transmembrane pressure of 2,500 KPa. A clear permeate passed from the membrane and a dark red retentate was produced rich in grape polyphenols.

A small quantity of malto dextrin was blended with the red juice and the concentrate was spray dried using a Niro spray drier. A rich red brown powder was produced. contiaing a rich source of grape polyphenols.

### Specific Example 4

### Solvent of the Grapes

Red grapes contain a range of poly phenols including anthocyanins and resvertarol. Red grape powder was charged into the extraction column of a supercritical carbon dioxide extractor. C02 supply from cylinder was fed to a high pressure pump and passed into the high pressure extraction column.

Optimised Supercritical extraction conditions.

| | | |
|---|---|---|
| Sample | 30 | grams |
| Extraction vessel | 50 | ml |
| Pressure | 420 | bar |
| Flow rate gas | 4 | l/min |
| Temperature | 60 | C. |

An oleo resin was collected in the separation vessel after a pressure reducing valve down to 60 bar.

### Specific Example 5

### Carrots

100 kg of fresh washed carrots were purchased direct from the markets.

The surface of the carrots were washed by a high pressure water spray and then in a tank of ozone water. The residence time in the ozone water tank was five minutes and the ozone level was maintained at 2 mg per litre.

The carrots were dewatered on a vibrating conveyor befor being elevated into a stainless steel hammer mill.

The hammermill was a commercial unit with a capacity of 1,000 Kg per hr and powered by a 4 kW electric motor. The rotational speed of the swing hammers was 2,800 rpm.

The carrot mulch passed through the screen into a stainless steel screw press which extracted a bright orange juice. The waste fibre had almost no residual colour.

Weighing of carrot feed and fibre showed a 50% yield of carrot juice. The juice passed to a Silverson L4RT high sheer mixer fitted with a square hole disintegrating head. The variable speed control was set at a rotational speed of 6,000 rpm as shown on the tachometer. High sheer was maintained for three minutes per batch at a temperature of 23 C.

The juice was then passed through a rotating wedge bar strainer with 100 micron openings. The strained juice passed to a membrane separation plant using a Nanofiltration spiral wound cartridge from Osmonics U.S.A. Membrane pore size one nanometre. The juice was concentrated by pumping at high cross flow through the membrane cartridge at 2,500 kPa pressure.

A clear permeate flowed from the membrane and the retentate turned from orange to brown as the B carotne became more concentrated.

The juice concentrate was dried using a Niro spray drier. An addition of 55 on solids of maltodextrin was added to the juice before spray drying. The drier operated with a 4 kW electric heater with an inlet air temperature of 200 C and and exit air temperature of 75 C. Feed was pumped into the atomiser nozzle using a peristaltic pump. The fine carrot powder was separated in a cyclone into the collecting chamber.

### Specific Example 6

### Solvent Extraction of Carrot Extract

The carrot powder from Example 5 was filled into an extraction vessel on a supercritical carbon dioxide extractor.

Carbon dioxide was released from a cylinder to a high pressure pump which also had a precooler. The pressure in the extraction cylinder was progressively ramped up to 250 bar and the flow rate set. The operating temperature was 40 C. At this temperature the Carbon dioxide was in a supercritical state and acted as a lipophilic solvent. The solvent CO2 passes through the column of carrot powder and then through the reducing valve until the pressure dropped to 60 bar. At this temperature the oleo resin was released into the separating vessel. The oleo resin was a rich orange brown showing a high level of B carotene.

### Specific Example 7

### A Grass

A crop of Alfalfa was harvested using a forage harvester and the harvested crop was brought directly to the processing facility.

The Alfalfa was washed with high pressure water spray and transferred to a conveyor belt feeding a hammer mill. The leaf was milled through a 5 mm screen and fed directly into a belt press.

The belt press was a commercial unit with a metre wide belt. At a feed rate of one tonne per hour the belt press delivered a very bright green juice at a rate of 500 Kg per hour.

The juice was subjected to high sheer using a Silverson high sheer mixer. A temperature of 25° C. was maintained while the stirrer operated with a batch time of three minutes. The Silverson mixer used was a BX model with a 0.75 kW motor and a speed of rotation of 3,000 rpm.

The unit was fitted with the standard disintegrator head.

The juice was passed through a 180 micron screen to the 200 litre feed tank of the membrane separation plant.

The custom built membrane plant used a 100 mm diameter spiral wound nanofiltration cartridge supplied by Desal, California U.S.A. Nominal pore size one nanometer. Typical rejection of 95% of molecules over 300 daltons.

| | | | | | | | | | Permeate | |
|---|---|---|---|---|---|---|---|---|---|---|
| Membrane | | | | Retentate | Feed | | Permeate | | Volume | |
| Pressure | | Time min | Temp Cel | Brix | Conductivity | | Conductivity | | Collected | |
| 2,500 | KPa | 0 | 20 | 5.5 | 5.1 | mS | 3.49 | mS | 20 | litres |
| 2,500 | | 10 | 24 | 6.4 | | | 4.14 | | 20 | l |
| 2,500 | | 20 | 27 | 7.5 | | | 4.16 | | 20 | l |
| 2,500 | | 30 | 35 | 11.5 | | | 4.54 | | 20 | l |
| 2,500 | | 40 | 40 | 15.0 | Final 9.1 | mS | 5.36 | | 20 | l |

The rich green juice was concentrated by membrane by a factor of 5:1.

Examination of the membrane permeate showed that it was water clear so that no green components passed through the membrane.

The conductivity of the nanofiltration permeate rose from 3.39 mili siemens to 5.36 mili siemens demonstrating the large quantity of mainly monovalent ions that were removed in the permeate.

Dry substance analysis was performed on the feed juice and the final membrane retentate.

Feed solids 8.3%

Final retentate concentrate solids=17.1%

Solids were determined by oven drying for 2 hrs at 95 C.

Microscopic examination of the concentrate showed fine green particles and a very bright green solution compared with the clear permeate.

Feed conductivity 5.1 mS Final concentrate conductivity 9.1 mS also demonstrating a moderate rise in minerals despite the 5:concentration factor.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A method to provide a concentrate obtained from plant material, the method comprising at least partially extracting juice from the plant material, subjecting at least some of the extracted juice to shear conditions to at least partially open plant cellular material, and removing at least some of the water to provide a concentrate.

2. The method as claimed in claim 1, wherein the concentrate is dried to a solid flowable material.

3. The method as claimed in claim 1, wherein the concentrate is extracted with a solvent to produce an extracted material.

4. The method as claimed in claim 2, wherein the flowable material is extracted with a solvent to produce an extracted material.

5. The method as claimed in claim 3, wherein the solvent is supercritical carbon dioxide.

6. The method as claimed in claim 4, wherein the solvent is supercritical carbon dioxide.

7. The method as claimed in claim 4, wherein a filler is added prior to solvent extraction.

8. The method as claimed in claim 1, wherein the water is removed using a semipermeable membrane.

9. The method as claimed in claim 8, wherein the semipermeable membrane is a nanofiltration membrane.

10. The method as claimed in claim 1, wherein the concentrate contains phytonutrients.

11. The method as claimed in claim 1, wherein the plant material is selected from the group consisting of fruits, vegetables, spices, herbs, grasses.

12. A solid flowable material made by the method of claim 9.
